(19) United States
(12) Patent Application Publication
(10) Pub. No.: US 2010/0047291 A1
(43) Pub. Date: Feb. 25, 2010

(54) COSMETIC FORMULATIONS WITH IR PROTECTION

(76) Inventors: Veronika Hochstein, Bruchsal (DE); Christoph Schmidt, Kriftel (DE); Sabine Schoen, Herten (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/993,607

(22) PCT Filed: Jun. 21, 2006
(86) PCT No.: PCT/EP2006/005949

§ 371 Date: Dec. 21, 2007

(30) Foreign Application Priority Data
Jun. 23, 2005 (DE) 10 2005 029 647.5

(51) Int. Cl. A61K 8/02; A61K 8/25; A61Q 17/04

(52) U.S. Cl. 424/401; 424/59

(57) ABSTRACT

The present invention relates to cosmetic formulations with IR protection which are distinguished by the fact that they comprise functional pigments. The invention furthermore relates to cosmetic formulations which, besides the IR pigment, comprise colorants, fillers, cosmetic active compounds and cosmetic raw materials and auxiliaries, and to the use thereof in care and decorative cosmetics.

The present invention relates to cosmetic formulations with IR protection which are distinguished by the fact that they comprise functional pigments. The invention furthermore relates to cosmetic formulations which, besides the IR pigment, comprise colorants, fillers, cosmetic active compounds and cosmetic raw materials and auxiliaries, and to the use thereof in care and decorative cosmetics.

About 44% of the energy incident on the earth's surface from the sun is in the infrared region. This is relatively long-wavelength (800 to about 3000 nm) and thus low-energy radiation. Infrared does not cause any direct biochemical changes in the cell structures of the skin (no electron excitation of the molecules) and also does not cause any photobiological damage. The penetration capacity into deeper skin layers as far as the subcutis should be emphasised; infrared lamps are therefore employed for the treatment of deep-lying inflammation, for relieving tension or for influencing specific diseases. Dilation of the blood vessels causes warming of the skin, which results in increased release of moisture. After sunbathing, the loss of moisture must be compensated for by suitable face creams or body lotions. Long-lasting, repeated, high-dose exposure to infrared rays can result in heat erythema or in extreme cases even in heat cancer. Infra-red is also said to cause denaturing and deactivation of important repair enzymes. The proportion and intensity of infrared rays during sunbathing are, inter alia, partly responsible for chronic skin damage (accelerated skin ageing, skin cancer). There is thus a demand for cosmetic products, such as, for example, sunscreens, which provide reliable protection against skin-damaging IR rays. High infrared protection is offered by water. Sun-screens based on emulsions with a high water content (lotions, creams) or hydrogels cool the skin not only through the evaporation effect, but also, in particular, by absorption of the infrared rays. In the case of aqueous application forms, however, the infrared protection is not long-lasting since the water evaporates.

The object of the present invention was therefore to provide cosmetic formulations which offer long-lasting and good protection against sunlight in the IR wavelength region (>700 nm).

Surprisingly, it has now been found that colour-neutral, transparent multi-layered pigments based on flake-form substrates act as light-protection filters in the wavelength region >700 nm (IR region) in cosmetic formulations. The IR pigments are colour-neutral and are therefore highly suitable in care cosmetics, but also in combination with other colorants in decorative cosmetics. In the present application, “substantially colour-neutral” means that the IR pigments have C values of <30, preferably <20, in particular <15 [C=\sqrt{(a²+b²)}=chroma=distance of the measurement point from the achromatic point a=b=0 in the a/b plane, measured using an ETA Plus calorimeter at a steep measurement angle in the vicinity of gloss (light incidence 75°/measurement 95°; 90°=perpendicular) on a black background].

The present invention thus relates to cosmetic formulations which comprise, as IR protection, a substantially colour-neutral, transparent multi-layered pigment based on flake-form substrates which has a layer package comprising high- and low-refractive-index layers on the surface.

The IR pigments prevent excessive warming of the skin since they have high transparency in the visible region of light (>700 nm) and high reflectivity in the NIR region. Pigments of this type are known, for example, from DE 196 18 569.

The IR pigments are based on a flake-form, transparent, colourless matrix, for example comprising mica (synthetic or natural), SiO₂ flakes, glass flakes, Al₂O₃ flakes, polymer flakes, and generally have a thickness between 0.2 and 5 μm, in particular between 0.4 and 2.0 μm. The size in the two other dimensions is usually between 1 and 300 μm, preferably between 2 and 100 μm and in particular between 5 and 70 μm.

The coating of the substrate flakes is carried out by forming a layer structure consisting of alternating high- and low-refractive-index layers. The layers on the flake-form substrate are preferably

(A) a colourless coating having a refractive index of n>1.8 and a layer thickness of 50-350 nm,

(B) a colourless coating having a refractive index of n≦1.8 and a layer thickness of 50-500 nm,

(C) a colourless coating having a refractive index of n>1.8 and a layer thickness of 20-350 nm,

optionally

(D) an outer protective layer.

The layer thicknesses (A) (B) (C) may be identical or different. The thicknesses of layers (A) and (C) are preferably in the range 20-350 nm, in particular 50-250 nm and very particularly preferably 70-150 nm. Layer (B) preferably has layer thicknesses of 50-500 nm, in particular 100-200 nm and very particularly preferably 130-180 nm.

Layers (A) and (C) preferably consist of TiO₂, ZrO₂, SnO₂, Fe₂O₃, Cr₂O₃, ZnO, BiOCl, or mixtures or combinations thereof. Layers (A) and (C) may be identical or different. Layers (A) and (C) are particularly preferably colourless layers, in particular TiO₂ layers. The TiO₂ here can be in the rutile or anatase modification, preferably rutile. The latter is generally achieved by coating, prior to the TiO₂ coating, with a layer of SnO₂ or Fe₂O₃. The layer thicknesses of the SnO₂ or Fe₂O₃ layer are preferably <50 nm, in particular <10 nm.

Suitable colourless, low-refractive-index materials which are suitable for coating (B) are preferably metal oxides or the corresponding oxide hydrates, such as, for example, SiO₂, Al₂O₃, AlO(OH), B₂O₃, MgF₂, MgSiO₃ or a mixture of the said metal oxides. Layer (B) preferably consists of SiO₂, MgF₂ or Al₂O₃ or mixtures thereof.

The layer package very particularly preferably consists of TiO₂—SiO₂—TiO₂.

Preferred IR pigments have the following structure:

mica flake+TiO₂ layer+SiO₂ layer+TiO₂ layer

SiO₂ flake+TiO₂ layer+SiO₂ layer+TiO₂ layer

Al₂O₃ flake+TiO₂ layer+SiO₂ layer+TiO₂ layer

glass flake+TiO₂ layer+SiO₂ layer+TiO₂ layer

mica flake+TiO₂/Fe₂O₃ layer+SiO₂ layer+TiO₂ layer

SiO₂ flake+TiO₂/Fe₂O₃ layer+SiO₂ layer+TiO₂ layer

Al₂O₃ flake+TiO₂/Fe₂O₃ layer+SiO₂ layer+TiO₂ layer

glass flake+TiO₂/Fe₂O₃ layer+SiO₂ layer+TiO₂ layer

mica flake+TiO₂ layer+SiO₂ layer+TiO₂/Fe₂O₃ layer

SiO₂ flake+TiO₂ layer+SiO₂ layer+TiO₂/Fe₂O₃ layer

Al₂O₃ flake+TiO₂ layer+SiO₂ layer+TiO₂/Fe₂O₃ layer

glass flake+TiO₂ layer+SiO₂ layer+TiO₂/Fe₂O₃ layer

mica flake+TiO₂/Fe₂O₃ layer+SiO₂ layer+TiO₂/Fe₂O₃ layer

SiO₂ flake+TiO₂/Fe₂O₃ layer+SiO₂ layer+TiO₂/Fe₂O₃ layer

Al₂O₃ flake+TiO₂/Fe₂O₃ layer+SiO₂ layer+TiO₂/Fe₂O₃ layer

glass flake+TiO₂/Fe₂O₃ layer+SiO₂ layer+TiO₂/Fe₂O₃ layer

mica flake+Cr₂O₃ layer+SiO₂ layer+TiO₂ layer

SiO₂ flake+Cr₂O₃ layer+SiO₂ layer+TiO₂ layer

Al₂O₃ flake+Cr₂O₃ layer+SiO₂ layer+TiO₂ layer

glass flake+Cr₂O₃ layer+SiO₂ layer+TiO₂ layer

mica flake+TiO₂ layer+SiO₂ layer+Cr₂O₃ layer

SiO₂ flake+TiO₂ layer+SiO₂ layer+Cr₂O₃ layer

Al₂O₃ flake+TiO₂ layer+SiO₂ layer+Cr₂O₃ layer

glass flake+TiO₂ layer+SiO₂ layer+Cr₂O₃ layer

Layers of Fe₂O₃ or Fe₂O₃/TiO₂ mixtures, Cr₂O₃ are particularly suitable, for example, for the production of skin colours, which also meet decorative demands (“bifunctional make-up”).

The incorporation of ZnO layers or ZnO interlayers or aftercoating with ZnO additionally enables a UV filter action to be introduced. Interlayers of this type have layer thicknesses of 1-200 nm, preferably 2-100 nm and very particularly preferably 5-50 nm.

Multilayered pigments of this type preferably exhibit the following layer sequences:

substrate+TiO₂+ZnO+SiO₂+TiO₂

substrate+ZnO+TiO₂+SiO₂+TiO₂

substrate+ZnO+TiO₂+SiO₂+TiO₂+ZnO

substrate+TiO₂+SiO₂+ZnO+TiO₂

substrate+TiO₂+ZnO+SiO₂+TiO₂+ZnO

Instead of ZnO, it is also possible here to use a mixture of SiO₂ and ZnO or a zinc silicate prepared therefrom, where the mixing ratio of SiO₂ to ZnO is unlimited and is dependent on the requirements of the IR pigment or on the needs of the preparation process. If desired, a multistep preparation process with intermittent isolation and calcination of intermediates can also be selected.

The metal-oxide layers on the substrate of the IR multilayered pigment are preferably applied by wet-chemical methods, it being possible to use the wet-chemical coating methods developed for the preparation of pearlescent pigments. Methods of this type are described, for example, in DE 14 67 468, DE19 59 988, DE 20 09 566, DE 22 14 545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 35 017 and in further patent documents and other publications known to the person skilled in the art.

In wet coating, the substrate particles are suspended in water, and one or more hydrolysable titanium salts are added at a pH which is suitable for hydrolysis, which is selected so that the metal oxides or metal oxide hydrates are precipitated directly onto the flakes without significant secondary precipitation occurring. The pH is usually kept constant by simultaneous metered addition of a base and/or acid. The pigments are subsequently separated off, washed and dried at 50-150° C. and optionally calcined for 0.1-3 h, where the calcination temperature can be optimised with respect to the particular coating present. In general, the calcination temperatures are between 250 and 1000° C., preferably between 350 and 950° C.

The coating can furthermore also be carried out by gas-phase coating in a fluidised-bed reactor, where, for example, the processes proposed in EP 0 045 851 A1 and EP 0 106 235 A1 for the preparation of pearlescent pigments can be used correspondingly.

In order to achieve specific colour effects, finely divided particles in the nanometre size range may additionally be introduced into the high- or low-refractive-index metal-oxide layers. Suitable for this purpose have proven to be, for example, finely divided ZnO, finely divided TiO₂ or finely divided carbon (for example through decomposition of organic substances, such as, for example, sugars, dextrin, etc.) or carbon black having particle sizes in the range 10-250 nm. The light-scattering properties of such particles enable the gloss and hiding power of the IR multilayered pigments to be influenced specifically.

The IR pigments may also be provided with a protective layer (layer D) in order to increase the light, weather and chemical stability or in order to increase the compatibility in various media. Suitable aftercoatings or after-treatments are, for example, the processes described in German Patent 22 15 191, DE-A 31 51 354, DE-A 32 35 017 or DE-A 33 34 598. This aftercoating further increases the chemical stability or simplifies handling of the pigment, in particular incorporation into various media. In order to improve the wettability, dispersibility and/or compatibility with the application media, functional coatings of Al₂O₃ or ZrO₂ or mixtures or mixed phases thereof can be applied to the pigment surface. Furthermore, organic or combined organic/inorganic aftercoatings are possible, for example with silanes, as described, for example, in EP 0090259, EP 0 634 459, WO 99/57204, WO 96/32446, WO 99/57204, U.S. Pat. No. 5,759,255, U.S. Pat. No. 5,571,851, WO 01/92425 or in J. J. Ponjee, Philips Technical Review, Vol. 44, No. 3, 81 ff. and P. H. Harding, J. C. Berg, J. Adhesion Sci. Technol. Vol. 11, No. 4, pp. 471-493.

The IR pigments are simple and easy to handle. The pigments can be incorporated into the cosmetic formulation by simple stirring-in. Complex grinding and dispersal of the pigments is not necessary.

The cosmetic formulations according to the invention preferably comprise colorants and/or fillers besides the IR multilayered pigment.

The cosmetic formulation according to the invention may comprise all organic and inorganic colorants (of natural or synthetic origin) or fillers that are known to the person skilled in the art, which preferably have a particle size of 0.001 to 10 μm, preferably 0.01 to 1 μm. The colorants preferably have a flake-form, spherical or needle-shaped particle shape. The fillers are preferably in the form of flake-form or spherical powders.

Besides the IR pigment, preferred cosmetic formulations comprise, in particular, a pearlescent pigment as colorant. The pearlescent pigments used are pigments based on flake-form, transparent or semitransparent substrates comprising, for example, phyllosilicates, such as, for example, natural mica, synthetic mica, talc, sericite, kaolin, aluminium oxide, silicon dioxide, glass or other silicate materials, which are coated with coloured or colourless metal oxides, such as, for example, TiO₂, titanium suboxides, titanium oxynitrides, Fe₂O₃, Fe₃O₄, SnO₂, Cr₂O₃, ZnO, CuO, NiO and other metal oxides, alone or in a mixture in a uniform layer or in successive layers. Pearlescent pigments are known, for example, from the German patents and patent applications 14 67 468,19 59 998, 20 09 566, 22 14 454, 22 15 191, 22 44 298, 23 13 331, 25 22 572, 31 37 808, 31 37 809, 31 51 343, 31 51 354, 31 51 355, 32 11 602, 32 35 017 and P 38 42 330 and are commercially available, for example, under the brands Timiron , Ronastar , Xirona®, Colorona®, Dichrona from Merck KGaA or Rona. Particularly preferred cosmetic formulations comprise TiO₂—, Fe₂O₃/TiO₂- and Fe₂O₃-coated mica flakes, SiO₂ flakes, Al₂O₃ flakes or glass flakes. Very particularly preferred pigments are glass flakes which have an SiO₂ layer and a TiO₂ and/or Fe₂O₃ layer on the surface.

The TiO₂ layers here can be in the anatase or rutile modification, preferably rutile. The latter is generally achieved by coating, prior to the TiO₂ coating, with a layer of SnO₂ or Fe₂O₃. The layer thicknesses of the SnO₂ or Fe₂O₃ layer are preferably <50 nm, in particular <10 nm.

Preference is furthermore given to TiO₂- and/or Fe₂O₃-coated SiO₂ or Al₂O₃ flakes. The coating of the coloured or colourless SiO₂ flakes with one or more metal oxides can be carried out, for example, as described in WO 93/08237 (wet-chemical coating) or DE-A 196 14637 (CVD method). The synthetically prepared SiO₂ flakes are preferably colourless, i.e. undoped SiO₂ flakes.

Suitable flake-form colorants are, in particular, pearlescent pigments, in particular based on mica, SiO₂ flakes, Al₂O₃ flakes or glass flakes, which are covered with one or more metal-oxide layers, metal-effect pigments (Al flakes, bronzes), optically variable pigments (OVPs), liquid-crystal polymer pigments (LCPs) or holographic pigments.

Preferred multilayered pigments having high gloss, pure interference colours and/or a strong colour flop have the following layer sequences:

mica flake+TiO₂+SiO₂+TiO₂

mica flake+Fe₂O₃+SiO₂+TiO₂

mica flake+TiO₂+SiO₂+TiO₂/Fe₂O₃

mica flake+TiO₂/Fe₂O₃+SiO₂+TiO₂/Fe₂O₃

mica flake+TiO₂/Fe₂O₃+SiO₂+TiO₂

mica flake+TiO₂/Fe₂O₃+SiO₂+Fe₂O₃

mica flake+Fe₂O₃+SiO₂+TiO₂/Fe₂O₃

Al₂O₃ flake+TiO₂+SiO₂+TiO₂

Al₂O₃ flake+Fe₂O₃+SiO₂+TiO₂

Al₂O₃ flake+TiO₂+SiO₂+TiO₂/Fe₂O₃

Al₂O₃ flake+TiO₂/Fe₂O₃+SiO₂+TiO₂/Fe₂O₃

Al₂O₃ flake+TiO₂/Fe₂O₃+SiO₂+TiO₂

Al₂O₃ flake+TiO₂/Fe₂O₃+SiO₂+Fe₂O₃

Al₂O₃ flake+Fe₂O₃+SiO₂+TiO₂/Fe₂O₃

SiO₂ flake+TiO₂+SiO₂+TiO₂

SiO₂ flake+Fe₂O₃+SiO₂+TiO₂

SiO₂ flake+TiO₂+SiO₂+TiO₂/Fe₂O₃

SiO₂ flake+TiO₂/Fe₂O₃+SiO₂+TiO₂/Fe₂O₃

SiO₂ flake+TiO₂/Fe₂O₃+SiO₂+TiO₂

SiO₂ flake+TiO₂/Fe₂O₃+SiO₂+Fe₂O₃

SiO₂ flake+Fe₂O₃+SiO₂+TiO₂/Fe₂O₃

glass flake+TiO₂+SiO₂+TiO₂

glass flake+Fe₂O₃+SiO₂+TiO₂

glass flake+TiO₂+SiO₂+TiO₂/Fe₂O₃

glass flake+TiO₂/Fe₂O₃+SiO₂+TiO₂/Fe₂O₃

glass flake+TiO₂/Fe₂O₃+SiO₂+TiO₂

glass flake+TiO₂/Fe₂O₃+SiO₂+Fe₂O₃

glass flake+Fe₂O₃+SiO₂+TiO₂/Fe₂O₃

glass flake+SiO₂+TiO₂+SiO₂+TiO₂

glass flake+SiO₂+Fe₂O₃+SiO₂+TiO₂

glass flake+SiO₂+TiO₂+SiO₂+TiO₂/Fe₂O₃

glass flake+SiO₂+TiO₂/Fe₂O₃+SiO₂+TiO₂/Fe₂O₃

glass flake+SiO₂+TiO₂/Fe₂O₃+SiO₂+TiO₂

glass flake+SiO₂+TiO₂/Fe₂O₃+SiO₂+Fe₂O₃

glass flake+SiO₂+Fe₂O₃+SiO₂+TiO₂/Fe₂O₃

If the cosmetic formulation according to the invention comprises an organic coloured pigment, this is preferably selected from the group of the quinacridones, xanthenes, indigoids, cyanines, antocyans, indanthrenes, monoazo, bisazo, trisazo colorants, triphenylmethanes, quinophthalones, anthraquinones, phthalocyanines, carotinoids or mixtures thereof.

The preferred spherical colorants include, in particular, TiO₂, coloured SiO₂, CaSO₄, iron oxides, chromium oxides, carbon black, organic coloured pigments, such as, for example, anthraquinone pigments, quinacridone pigments, diketopyrrolopyrrole pigments, phthalocyanine pigments, azo pigments, isoindoline pigments. The needle-shaped pigments are preferably BiOCl, coloured glass fibres, α-FeOOH, organic coloured pigments, such as, for example, azo pigments, β-phthalocyanine Cl Blue 15,3, Cromophtal Yellow 8GN (Ciba-Geigy), Irgalith Blue PD56 (Ciba-Geigy), azomethine/copper complex Cl Yellow 129, Irgazine Yellow 5GT (Ciba-Geigy).

The formulations according to the invention comprise the IR pigment and further colorants in amounts of 0.5-100% by weight, preferably 10-80% by weight, in particular 5-70% by weight, depending on the area of use.

The ratio of IR pigment to further colorants is preferably 30:1 to 1:10, in particular 20:1 to 1:5, particularly preferably 5:1 to 1:5, depending on the respective area of use.

The cosmetic formulation according to the invention may furthermore comprise commercially available fillers. Fillers which may be mentioned are, for example, polymethyl methacrylate, methyl methacrylate cross-polymer, natural and synthetic mica, nylon powder, for example nylon 12, pure or filled melamine resins, talc, clay, glass powder (in flake or bead form), kaolin, SiO₂ (preferably as flakes or in bead form), oxides or hydroxides of aluminium, magnesium, calcium, zinc, BiOCl, barium sulfate, calcium sulfate, basic alkaline-earth metal carbonates, such as, for example, calcium carbonate or magnesium carbonate, carbon, and physical or chemical combinations of these substances. There are no restrictions regarding the particle shape of the filler. In accordance with requirements, it can be, for example, flake-form, spherical or needle-shaped. Particularly preferred fillers and/or colorants are coated or uncoated SiO₂ beads. SiO₂ beads coated with one or more metal oxides are known, for example, from EP 0 803 550 A2. The said fillers are commercially available, such as, for example, Mica M, Satin Mica, Silk Mica, Micronaphere M, Ronasphere, Ronasphere LDP. Flake-form BiOCl is commercially available, for example, as a slurry, paste, powder, in suspension, under the product names Biron®, Mibiron®, Nailsyn® from Merck KGaA or Rona.

Preferred cosmetic formulations according to the invention comprise a filler mixture, for example comprising BiOCl+mica+SiO₂, mica+SiO₂, BiOCl +glass powder (for example comprising calcium aluminium borosilicate), mica+methyl methacrylate crosspolymer, butyl methacrylate-ethylene glycol methacrylate copolymer. Filler mixtures of this type are marketed, for example, by Presperse under the brands SM, SP, Sphericite or Pearlsil.

The cosmetic formulations according to the invention may of course comprise any type of cosmetic raw material and auxiliary. These include, inter alia, oils, fats, waxes, film formers, preservatives and auxiliaries which generally determine the applicational properties, such as, for example, thickeners and rheological additives, such as, for example, bentonites, hectorites, silicon dioxides, Ca silicates, gelatines, high-molecular-weight carbohydrates and/or surface-active auxiliaries, etc.

The cosmetic formulations according to the invention may belong to the lipophilic, hydrophilic or hydrophobic type. In the case of heterogeneous formulations having discrete aqueous and non-aqueous phases, the IR pigments may be present in in each case only one of the two phases or alternatively distributed over both phases.

The pH values of the formulations can be between 1 and 14, preferably between 2 and 11 and particularly preferably between 5 and 8.

The cosmetic formulation according to the invention may furthermore also comprise cosmetic active compounds, such as, for example, insect repellents, UV A/BC protection filters (for example OMC, B3, MBC), antiageing active compounds, vitamins and derivatives thereof (for example vitamins A, C, E etc.), self-tanning agents (for example DHA, erythrulose, inter alia), and further cosmetic active compounds, such as, for example, bisabolol, LPO, ectoin, emblica, allantoin, bioflavonoids and derivatives thereof.

The invention relates, in particular, to cosmetic formulations which, besides the IR pigment, comprise at least one constituent selected from absorbents, astringents, antimicrobial substances, antioxidants, antiperspirants, antifoaming agents, antidandruff active compounds, antistatics, binders, biological additives, bleaches, chelating agents, deodorants, emollients, emulsifiers, emulsion stabilisers, colorants, dyes, humectants, film formers, fillers, fragrances, flavours, insect repellents, preservatives, corrosion-protection agents, cosmetic oils, solvents, oxidants, vegetable constituents, buffer substances, reducing agents, surfactants, propellant gases, opacifiers, UV filters, UV absorbers, denaturing agents, viscosity regulators, perfume and vitamins.

Owing to its colour neutrality, the IR pigment can advantageously be employed in decorative and care cosmetics. The cosmetic formulations according to the invention can be used, for example, in nail varnishes, compact powders, shampoos, loose powders and gels, skin creams, skin lotions, sunscreens, skin-tanning agents, insect repellents, hair-care products, such as, for example, a hair cure, a rinse or a conditioner, permanent-wave products, hair-setting products, such as, for example, a hair set, a hair spray, a hair lacquer, a hair gel or a hair wax, hair colorants, a tint, a face make-up, such as, for example, a tinted day cream, a powder cream, a face powder, cream make-up or a rouge, eye make-up, such as, for example, an eye shadow, a mascara, a kohl pencil, an eyeliner or an eyebrow pencil, a lip-care product, a lipstick, a lip gloss or a lip liner, a nail-care product, such as, for example, a nail varnish, a nail hardener or a nail-care cream.

Further embodiments and combinations are revealed by the examples and claims.

The following examples are intended to explain the invention in greater detail without limiting it:

### I. PREPARATION EXAMPLES

### Example 1

100 g of mica having a particle size of 10-60 μm are suspended in 2 litres of deionised water and heated to 80° C. with vigorous stirring. A solution of 3 g of SnCl₄×5 H₂O and 10 ml of hydrochloric acid (37%) in 90 ml of deionised water is metered into this mixture at pH=2.0. An amount of 500 ml of TiCl₄ solution (400 g of TiCl₄/I) is subsequently metered in at a pH of 5 1.8. The pH is then adjusted to 7.5 using sodium hydroxide solution (32%), and a solution of 327 g of sodium water-glass solution (26% by weight of SiO₂) in 327 g of deionised water is metered in at this pH. During this addition, the pH is kept constant using hydrochloric acid (10% by weight of HCl). A solution of 3 g of SnCl₄×5 H₂O and 10 ml of hydrochloric acid (32%) in 90 ml of deionised water is subsequently metered in at pH=2.0. Finally, 500 ml of TiCl₄ solution (400 g of TiCl₄/I) are metered in at a pH of 1.8.

During the addition of the SnCl₄×5 H₂O solutions and TiCl₄ solutions, the pH is in each case kept constant using NaOH solution (32%).

For work-up, the pigment is filtered off, washed with 20 l of deionised water, dried at 110° C. and calcined at 850° C. for 0.5 h.

The pigment is substantially colourless and reflects infrared light in the range from about 750 to 1500 nm.

### Example 2

100 g of Al₂O₃ flakes having a particle size of 5-30 μm are suspended in 2 l of deionised water and heated to 80° C. with vigorous stirring. A solution of 11.5 g of SnCl₄×5 H₂O and 38 ml of hydrochloric acid (37%) in 350 ml of deionised water is metered into this mixture at pH=2.0. An amount of about 460 ml of TiCl₄ solution (400 g of TiCl₄/I) is subsequently metered in at a pH of 1.8. The pH is then adjusted to 7.5 using sodium hydroxide solution (32%), and a solution of 230 g of sodium water-glass solution (26% by weight of SiO₂) in 230 ml of deionised water is metered in at this pH. During this addition, the pH is kept constant using hydrochloric acid (10%). A solution of 11.5 g of SnCl₄×5 H₂O and 38 ml of hydrochloric acid (32%) in 350 ml of deionised water is subsequently metered in at pH=2.0.

Finally, 460 ml of TiCl₄ solution (400 g of TiCl₄/I) are metered in at a pH of 1.8.

During the addition of the SnCl₄×5 H₂O solutions and TiCl₄ solutions, the pH is in each case kept constant using NaOH solution (32%).

For work-up, the pigment is filtered off, washed with 20 l of deionised water, dried at 110° C. and calcined at 850° C. for 30 min.

The pigment is substantially colourless and reflects infrared light in the range from about 700 to 1500 nm.

### Example 3

100 g of SiO₂ flakes having a particle size of 5-40 μm are suspended in 2 l of deionised water and heated to 80° C. with vigorous stirring. A solution of 11.5 g of SnCl₄×5 H₂O and 38 ml of hydrochloric acid (37%) in 350 ml of deionised water is metered into this mixture at pH=2.0. An amount of 445 ml of TiCl₄ solution (400 g of TiCl₄/I) is subsequently metered in at a rate of 2 ml/min at a pH of 1.8. The pH is then adjusted to 7.5 using sodium hydroxide solution (32%), and a solution of 322 g of sodium water-glass solution (26% by weight of SiO₂) in 322 g of deionised water is metered in at this pH. During this addition, the pH is kept constant using hydrochloric acid (10%). A solution of 11.5 g of SnCl₄×5 H₂O and 38 ml of hydrochloric acid (32%) in 350 ml of deionised water is subsequently metered in at pH=2.0. Finally, about 475 ml of TiCl₄ solution (400 g of TiCl₄/I) are metered in at a pH of 1.8.

During the addition of the SnCl₄×5 H₂O solutions and TiCl₄ solutions, the pH is in each case kept constant using NaOH solution (32%).

For work-up, the pigment is filtered off, washed with 20 l of deionised water, dried at 110° C. and calcined at 850° C. for 30 min.

The pigment is substantially colourless and reflects infrared light in the range from about 700 to 1500 nm, where two pronounced maxima of the reflection occur at about 820 nm and about 1200 nm.

### Example 4

200 g of calcium aluminium borosilicate flakes having a particle size of 20-199 μm and 100 g of NaCl are suspended in 2 l of deionised water and heated to 70° C. with vigorous stirring.

The pH is adjusted to 9.0 using sodium hydroxide solution (32% by weight of NaOH), and a solution of 154 g of sodium water-glass solution (26% by weight of SiO₂) in 154 g of deionised water is metered in at this pH. The pH is subsequently lowered to 1.8 using hydrochloric acid (10% by weight of HCl), and a solution of 5.2 g of SnCl₄×5 H₂O and 18 ml of hydrochloric acid (37%) in 180 ml of deionised water is metered in. An amount of about 285 ml of TiCl₄ solution (400 g of TiCl₄/I) is subsequently metered in at a pH of 1.2. The pH is then adjusted to 9.0 using sodium hydroxide solution (32%), and a solution of 192 g of sodium water-glass solution (26% by weight of SiO₂) in 192 ml of deionised water is metered in at this pH.

During this addition, the pH is kept constant using hydrochloric acid (10% by weight of HCl). A solution of 5.2 g of SnCl₄×5 H₂O and 18 ml of hydro-chloric acid (32%) in 180 ml of deionised water is subsequently metered in at pH=1.8. Finally, about 358 ml of TiCl₄ solution (400 g of TiCl₄/I) are metered in at a pH of 1.8.

During the addition of the SnCl₄×5 H₂O solutions and TiCl₄ solutions, the pH is in each case kept constant using NaOH solution (32%).

For work-up, the pigment is filtered off, washed with 10 l of deionised water, dried at 110° C. and calcined at 650° C. for 30 min.

The pigment is substantially colourless and reflects infrared light in the range from about 700 to 1500 nm.

### II. Use Examples

### Example A1

### Body Cream (O/W)

| Raw material | | INCI | [%] |
|---|---|---|---|
| Phase A | | | |
| IR-reflective pigment according | (1) | SILICA, CI 77891 (TITANIUM DIOXIDE), TIN | 5.00 |
| to Example 3 | | OXIDE | |
| Carbopol Ultrez 21 | (2) | ACRYLATES/C10-30 ALKYL ACRYLATE | 0.60 |
| | | CROSSPOLYMER | |
| Citric acid monohydrate | (1) | CITRIC ACID | 0.00 |
| Water, demineralised | | AQUA (WATER) | 37.60 |
| Phase B | | | |
| RonaCare ® allantoin | (1) | ALLANTOIN | 0.20 |
| 1,2-Propanediol | (1) | PROPYLENE GLYCOL | 3.00 |
| Water, demineralised | | AQUA (WATER) | 26.35 |
| Phase C | | | |
| Hostaphat KL 340 D | (3) | TRILAURETH-4-PHOSPHATE | 3.00 |
| Cetyl alcohol | (1) | CETYL ALCOHOL | 2.00 |
| Paraffin, liquid | (1) | PARAFFINUM LIQUIDUM (MINERAL OIL) | 10.00 |
| Cetiol V | (4) | DECYL OLEATE | 6.00 |
| Phenonip | (5) | PHENOXYETHANOL, BUTYLPARABEN, | 0.50 |
| | | ETHYLPARABEN, PROPYLPARABEN, | |
| | | METHYLPARABEN | |
| Phase D | | | |
| Triethanolamine | (1) | TRIETHANOLAMINE | 0.35 |
| Water, demineralised | | AQUA (WATER) | 3.50 |
| Phase E | | | |
| Vogue perfume oil | (6) | PERFUME | 0.10 |
| Germall 115 | (7) | IMIDAZOLIDINYL UREA | 0.30 |
| Water, demineralised | | AQUA (WATER) | 1.50 |

Preparation:

Disperse the IR pigment in the water of phase A. If necessary, acidify using a few drops of citric acid in order to reduce the viscosity. Scatter in the Carbopol with stirring. When completely dissolved, slowly stir in the pre-dissolved phase B. Heat phase A/B and phase C to 80° C., stir phase C into phase A/B, homogenise, neutralise using phase D, homogenise again and cool with stirring. At 40° C., cool phase E to room temperature with stirring.

Sources of Supply:

(1) Merck KGaA/Rona®

(2) Noveon

(3) Clariant GmbH

(4) Cognis GmbH

(5) Nipa Laboratorien GmbH

(6) Drom

(7) ISP Global Technologies

### Example A2

### Tinted Day Cream with IR and UV Protection (O/W)

| Raw material | | INCI | [%] |
|---|---|---|---|
| Phase A | | | |
| Eusolex ® 2292 | (1) | ETHYLHEXYL METHOXYCINNAMATE, BHT | 3.00 |
| Eusolex ® 4360 | (1) | BENZOPHENONE-3 | 3.00 |
| Eusolex ® HMS | (1) | HOMOSALATE | 5.00 |
| Eusolex ® OS | (1) | ETHYLHEXYL SALICYLATE | 5.00 |
| Arlacel 165 VP | (2) | GLYCERYL STEARATE, PEG-100 STEARATE | 3.00 |
| Montanov 68 | (3) | CETEARYL ALCOHOL, CETEARYL | 3.00 |
| | | GLUCOSIDE | |
| Dow Corning 345 | (4) | CYCLOMETHICONE | 0.50 |
| Eutanol G | (5) | OCTYLDODECANOL | 2.00 |
| Phenonip | (8) | PHENOXYETHANOL, BUTYLPARABEN, | 0.50 |
| | | ETHYLPARABEN, PROPYLPARABEN, | |
| | | METHYLPARABEN | |
| Phase B | | | |
| Eusolex ® T-2000 | (1) | TITANIUM DIOXIDE, ALUMINA, | 3.00 |
| | | SIMETHICONE | |
| Extender W | (1) | MICA, CI 77891 (TITANIUM DIOXIDE) | 4.00 |
| Microna ® Matte Yellow | (1) | MICA, CI 77492 (IRON OXIDES) | 2.00 |
| Microna ® Matte Orange | (1) | MICA, CI 77491 (IRON OXIDES) | 0.20 |
| Microna ® Matte Red | (1) | CI 77491 (IRON OXIDES), MICA | 0.20 |
| Microna ® Matte Black | (1) | CI 77499 (IRON OXIDES), MICA | 0.20 |
| IR-reflective pigment according | (1) | CI 77891 (TITANIUM DIOXIDE), MICA, SILICA, | 8.00 |
| to Example 1 | | TIN OXIDE | |
| Karion FP, liquid | (1) | SORBITOL | 5.00 |
| RonaCare ® allantoin | (1) | ALLANTOIN | 0.50 |
| Keltrol T | (6) | XANTHAN GUM | 0.20 |
| Germall 115 | (7) | IMIDAZOLIDINYL UREA | 0.30 |
| Water, demineralised | | AQUA (WATER) | 51.35 |

Preparation:

Disperse all constituents apart from the Keltrol T in the water of phase B. Scatter the Keltrol into phase B with stirring and heat to 80° C. after 15 minutes. Heat phase A to 75° C. Slowly stir in phase B and homogenise. Cool with stirring.

Sources of Supply:

(1) Merck KGaA/Rona®

(2) Uniqema

(3) Seppic

(4) Dow Corning

(5) Cognis GmbH

(6) C. P. Kelco

(7) ISP Global Technologies

(8) Nipa Laboratorien GmbH

### Example A3

### Shimmering Foundation

| Raw material | | INCI | [%] |
|---|---|---|---|
| Phase A | | | |
| Extender W | (1) | MICA, CI 77891 (TITANIUM DIOXIDE) | 9.00 |
| Microna ® Matte Yellow | (1) | MICA, CI 77492 (IRON OXIDES) | 4.00 |
| Microna ® Matte Red | (1) | CI 77491 (IRON OXIDES), MICA | 0.40 |
| Microna ® Matte Black | (1) | CI 77499 (IRON OXIDES), MICA | 0.30 |
| Timiron ® Supersheen MP-1001 | (1) | MICA, CI 77891 (TITANIUM DIOXIDE) | 4.50 |
| IR-reflective pigment according | (1) | CI 77891 (TITANIUM DIOXIDE), MICA, SILICA, | 8.00 |
| to Example 1 | | TIN OXIDE | |
| Phase B | | | |
| Blanose 7 HF | (2) | CELLULOSE GUM | 0.20 |
| Veegum | (3) | MAGNESIUM ALUMINUM SILICATE | 1.00 |
| Texapon K 1296 | (4) | SODIUM LAURYL SULFATE | 0.60 |
| Triethanolamine, extra pure | (1) | TRIETHANOLAMINE | 0.50 |
| Titriplex ® III | (1) | DISODIUM EDTA | 0.25 |
| Methyl 4-hydroxybenzoate | (1) | METHYLPARABEN | 0.15 |
| 1,2-Propanediol | (1) | PROPYLENE GLYCOL | 10.90 |
| Water, demineralised | | AQUA (WATER) | 39.95 |
| Phase C | | | |
| Isopropyl myristate | (4) | ISOPROPYL MYRISTATE | 8.00 |
| Paraffin, liquid | (1) | PARAFFINUM LIQUIDUM (MINERAL OIL) | 3.60 |
| Crodamol SS | (5) | CETYL ESTERS | 2.60 |
| Monomuls 60-35 C | (4) | HYDROGENATED PALM GLYCERIDES | 1.70 |
| Stearic acid | (1) | STEARIC ACID | 1.50 |
| Eusolex ® 6300 | (1) | 4-METHYLBENZYLIDENE CAMPHOR | 1.30 |
| Eusolex ® 4360 | (1) | BENZOPHENONE-3 | 0.50 |
| RonaCare ® tocopherol acetate | (1) | TOCOPHERYL ACETATE | 0.50 |
| Magnesium stearate | (1) | MAGNESIUM STEARATE | 0.10 |
| Phenonip | (8) | PHENOXYETHANOL, BUTYLPARABEN, | 0.50 |
| | | ETHYLPARABEN, PROPYLPARABEN, | |
| | | METHYLPARABEN | |
| Phase D | | | |
| Germall 115 | (7) | IMIDAZOLIDINYL UREA | 0.30 |
| Water, demineralised | | AQUA (WATER) | 1.50 |
| Phase E | | | |
| Perfume oil 200 529 | (6) | PERFUME | 0.20 |

Preparation:

Melt all constituents of phase C at about 75° C. and stir until everything has melted. Initially introduce the cold water of phase B, homogenise the Blanose using the Turrax, scatter in the Veegum, and re-homogenise. Warm to 75° C., and dissolve the other constituents therein with stirring. Stir in the ingredients of phase A. Add phase C at 75° C. with stirring and homogenise for 2 min. Cool the mass to 40° C. with stirring, add phases D and E. Cool further to room temperature with stirring and adjust to pH 6.0-6.5 (for example using citric acid solution).

Due to the addition of a pearlescent pigment, such as, for example, TIMIRON® Super Sheen MP-1001 (TiO₂-coated mica pigment having a particle size of 5-25 μm from Merck KGaA), this easily spreadable foundation leaves a silky sheen on the skin. The colour does not change after application.

Sources of Supply:

(1) Merck KGaA/Rona®

(2) Aqualon GmbH

(3) Vanderbilt

(4) Cognis GmbH

(5) Croda GmbH

(6) Fragrance Resources

(7) ISP Global Technologies

(8) Nipa Laboratorien GmbH

### Example A4

### Long-lasting Lip Gloss

| Raw material | | INCI | [%] |
|---|---|---|---|
| Phase A | | | |
| Xirona ® Volcanic Fire | (1) | CI 77891 (TITANIUM DIOXIDE), SILICA, MICA, | 5.00 |
| | | TIN OXIDE | |
| IR-reflective pigment according | (1) | CALCIUM ALUMINUM BOROSILICATE, CI | 7.00 |
| to Example 4 | | 77891, (TITANIUM DIOXIDE), SILICA, TIN | |
| | | OXIDE | |
| Phase B | | | |
| Indopol H 100 | (2) | POLYBUTENE | 30.00 |
| Jojoba Glaze LV | (3) | SIMMONDSIA CHINENSIS (JOJOBA), SEED | 20.00 |
| | | OIL, ETHYLENE/PROPYLENE/STYRENE | |
| | | COPOLYMER, BUTYLENE/ETHYLENE/- | |
| | | STYRENE COPOLYMER | |
| Jojoba Glaze HV | (3) | SIMMONDSIA CHINENSIS (JOJOBA), SEED | 10.00 |
| | | OIL, ETHYLENE/PROPYLENE/STYRENE | |
| | | COPOLYMER, BUTYLENE/ETHYLENE/- | |
| | | STYRENE COPOLYMER | |
| Castor oil | (4) | RICINUS COMMUNIS (CASTOR OIL) | 21.25 |
| Beeswax, white | (1) | CERA ALBA (BEESWAX) | 4.00 |
| Rouge Covapate W 3773 | (5) | RICINUS COMMUNIS (CASTOR OIL), | 0.40 |
| | | CI 15850 (D&C RED NO 6) | |
| Propyl 4-hydroxybenzoate | (1) | PROPYLPARABEN | 0.10 |
| Oxynex ® K liquid | (1) | PEG-8, TOCOPHEROL, ASCORBYL | 0.05 |
| | | PALMITATE, ASCORBIC ACID, CITRIC ACID | |
| Phase C | | | |
| Neosil CT 11 | (6) | SILICA | 2.00 |
| Fragrance Tendresse 75418C | (7) | PERFUME | 0.20 |

Preparation:

All constituents of phase B are weighed out together, heated to 80° C. and stirred well. Stir in the pigments of phase A, scatter in the Neosil with stirring, and finally add the perfume. All constituents of phase B are weighed out together, heated to 80° C. and stirred well. Transfer the homogeneous mixture into containers.

Sources of Supply:

(1) Merck KGaA/Rona®

(2) BP Lavera Sud

(3) Desert Whale

(4) Henry Lamotte GmbH

(5) Les Colorants Wackherr

(6) Ineos Silicas Limited

(7) Symrise

### Example A5

### Wrinkle-Reducing Day Cream (O/W)

| Raw material | | INCI | [%] |
|---|---|---|---|
| Phase A | | | |
| Ronasphere ® LDP | (1) | SILICA, CI 77891 (TITANIUM DIOXIDE), | 7.00 |
| | | CI 77491 (IRON OXIDES) | |
| IR-reflective pigment according | (1) | ALUMINA, CI 77891 (TITANIUM DIOXIDE), | 10.00 |
| to Example 2 | | SILICA, TIN OXIDE | |
| Veegum HV | (2) | MAGNESIUM ALUMINUM SILICATE | 1.00 |
| Karion F liquid | (1) | SORBITOL | 3.00 |
| Methyl 4-hydroxybenzoate | (1) | METHYLPARABEN | 0.18 |
| Water, demineralised | | AQUA (WATER) | 42.32 |
| Phase B | | | |
| Arlacel 165 VP | (3) | GLYCERYL STEARATE, PEG-100 STEARATE | 5.00 |
| Lanette O | (4) | CETEARYL ALCOHOL | 1.50 |
| Miglyol 812 N | (5) | CAPRYLIC/CAPRIC TRIGLYCERIDE | 7.00 |
| Shea butter, solid | (6) | BUTYROSPERMUM PARKII (SHEA BUTTER) | 2.00 |
| Cetiol SN | (4) | CETEARYL ISONONANOATE | 7.00 |
| Eutanol G | (4) | OCTYLDODECANOL | 7.50 |
| Emulgade PL 68/50 | (4) | CETEARYL ALCOHOL, CETEARYL | 2.00 |
| | | GLUCOSIDE | |
| Phase C | | | |
| Perfume oil 200 530 | (7) | PERFUME | 0.20 |
| Dow Corning 345 | (8) | CYCLOMETHICONE | 2.00 |
| Phenonip | (9) | PHENOXYETHANOL, BUTYLPARABEN, | 0.50 |
| | | ETHYLPARABEN, PROPYLPARABEN, | |
| | | METHYLPARABEN | |
| Phase D | | | |
| Water, demineralised | | AQUA (WATER) | 1.50 |
| Germall 115 | (10) | IMIDAZOLIDINYL UREA | 0.30 |
| Citric acid monohydrate | (1) | CITRIC ACID | 0.00 |

Preparation:

Warm phase B until the solution is clear. Disperse the Veegum in the water of phase A, add the remaining raw materials, heat to 80° C., and add phase B. Homogenise phase A/B. Cool to 40° C. with stirring and add phases C and D. Cool to room temperature and adjust to pH 6.0.

Sources of Supply:

(1) Merck KGaA/Rona®

(2) Vanderbilt

(3) Uniqema

(4) Cognis GmbH

(5) Sasol Germany GmbH

(6) H. Erhard Wagner GmbH

(7) Fragrance Resources

(8) Dow Corning

(9) Nipa Laboratorien GmbH

(10) ISP Global Technologies

### Example A6

### Shampoo

| Raw material | | INCI | [%] |
|---|---|---|---|
| Phase A | | | |
| IR-reflective pigment according | (1) | CI 77891 (TITANIUM DIOXIDE), MICA, SILICA, | 2.00 |
| to Example 1 | | TIN OXIDE | |
| Carbopol ETD 2020 | (2) | ACRYLATES/C10-30 ALKYL ACRYLATE | 0.90 |
| | | CROSSPOLYMER | |
| Water, demineralised | | AQUA (WATER) | 61.60 |
| Phase B | | | |
| Triethanolamine, extra pure | (1) | TRIETHANOLAMINE | 0.90 |
| Water, demineralised | | AQUA (WATER) | 10.00 |
| Phase C | | | |
| Plantacare 2000 UP | (3) | DECYL GLUCOSIDE | 20.00 |
| Texapon ASV 50 | (3) | SODIUM LAURETH SULFATE, SODIUM | 4.35 |
| | | LAURETH-8 SULFATE, MAGNESIUM | |
| | | LAURETH SULFATE, MAGNESIUM | |
| | | LAURETH-8 SULFATE, SODIUM OLETH | |
| | | SULFATE, MAGNESIUM OLETH SULFATE | |
| Bronidox L | (3) | PROPYLENE GLYCOL, 5-BROMO-5-NITRO- | 0.20 |
| | | 1,3-DIOXANE | |
| Perfume oil 200 524 | (4) | PERFUME | 0.05 |
| Dye solution (q.s.) | | | 0.00 |

Preparation:

For phase A, stir the IR pigment into the water. Acidify using a few drops of citric acid (10%) in order to reduce the viscosity, and slowly scatter in the Carbopol with stirring. When completely dissolved, slowly add phase B. The constituents of phase C are then added successively. Adjust the pH to 6.0-6.5.

Sources of Supply:

(1) Merck KGaA/Rona®

(2) Noveon

(3) Cognis GmbH

(4) Fragrance Resources

### Example A7

### Hair Styling Gel

| Raw material | | INCI | [%] |
|---|---|---|---|
| Phase A | | | |
| IR-reflective pigment according | (1) | SILICA, CI 77891 (TITANIUM DIOXIDE), TIN | 8.00 |
| to Example 3 | | OXIDE | |
| Carbopol Ultrez 21 | (2) | ACRYLATES/C10-30 ALKYL ACRYLATE | 0.90 |
| | | CROSSPOLYMER | |
| 2-Propanol | (1) | ISOPROPYL ALCOHOL | 20.00 |
| Water, demineralised | | AQUA (WATER) | 30.00 |
| Phase B | | | |
| Luviskol K 30 powder | (3) | PVP | 2.00 |
| Germaben II | (4) | PROPYLENE GLYCOL, DIAZOLIDINYL UREA, | 1.00 |
| | | METHYLPARABEN, PROPYLPARABEN | |
| Triethanolamine, extra pure | (1) | TRIETHANOLAMINE | 2.16 |
| Water, demineralised | | AQUA (WATER) | 35.94 |

Preparation:

Disperse the IR pigments in the water/propanol mixture of phase A, and scatter in the Carbopol with stirring. When completely dissolved, slowly stir in the pre-dissolved phase B.

Sources of Supply:

(1) Merck KGaA/Rona®

(2) Noveon

(3) BASF AG

(4) ISP Global Technologies

### Example A8

### Cream Conditioner

| Raw material | | INCI | [%] |
|---|---|---|---|
| Phase A | | | |
| Water | | AQUA (WATER) | 75.2 |
| IR-reflective pigment according | (1) | CI 77891 (TITANIUM DIOXIDE), MICA, SILICA, | 5.00 |
| to Example 1 | | TIN OXIDE | |
| Luviquat Hold | (2) | POLYQUATERNIUM-46 | 5.00 |
| Luviquat PQ 11 | (2) | POLYQUATERNIUM-11 | 2.00 |
| Butylene glycol | | BUTYLENE GLYCOL | 3.00 |
| Phase B | | | |
| Cremophor A 6 | (2) | CETEARETH-6 AND STEARYL ALCOHOL | 3.00 |
| Ammonyx 4 | (3) | STEARALKONIUM CHLORIDE | 3.00 |
| Lanette O | (4) | CETEARYL ALCOHOL | 2.00 |
| Eusolex 2292 | (1) | OCTYL METHOXYCINNAMATE | 0.10 |
| Phase C | | | |
| RonaCare ® tocopherol acetate | (1) | TOCOPHERYL ACETATE | 0.50 |
| RonaCare ® bisabolol | (1) | BISABOLOL | 0.10 |
| Perfume | | PERFUME | 0.10 |
| Germaben II | (5) | PROPYLENE GLYCOL, DIAZOLIDINYL UREA, | 1.00 |
| | | METHYLPARABEN, PROPYLPARABEN | |

Preparation:

Disperse the IR pigment in the water of phase A, and add the remaining raw materials. Stir after each addition and subsequently heat to 75° C. Mix the raw materials of phase B, heat to 75-80° C. and add to phase A. Mix until a homogeneous distribution is present. Add phase C at 45° C.

Sources of Supply:

(1) Merck KGaA/Rona®

(2) BASF AG

(3) Stepan

(4) Henkel KGaA

(5) ISP Global Technologies

### Example A9

### Shimmering Body Powder

| Raw material | | INCI | [%] |
|---|---|---|---|
| Phase A | | | |
| Ronastar ® Copper Sparks | (1) | CALCIUM ALUMINUM BOROSILICATE, | 10.00 |
| | | SILICA, CI 77891 (TITANIUM DIOXIDE), TIN | |
| | | OXIDE | |
| IR-reflective pigment according | (1) | MICA, SILICA, CI 77891 (TITANIUM DIOXIDE), | 30.00 |
| to Example 1 | | TIN OXIDE | |
| Phase B | | | |
| White clay | (1) | KAOLIN | 14.70 |
| Talc | (1) | TALC | 10.00 |
| Mica M | (1) | MICA | 15.00 |
| Silk mica | (1) | MICA | 9.50 |
| Ronaspher ® LDP | (1) | SILICA, CI 77891 (TITANIUM DIOXIDE), CI | 4.00 |
| | | 77491 (IRON OXIDES) | |
| Microna ® Matte Yellow | (1) | MICA, CI 77492 (IRON OXIDES) | 1.00 |
| Microna ® Matte Red | (1) | MICA, CI 77491 (IRON OXIDES) | 1.00 |
| Propyl 4-hydroxybenzoate | (1) | PROPYLPARABEN | 0.30 |
| Phase C | | | |
| Fitoderm | (2) | SQUALENE | 2.00 |
| Miglyol 812 N | (3) | CAPRYLIC/CAPRIC TRIGLYDERIDE | 2.00 |
| Perfume oil | | PERFUME | 0.30 |
| RonaCare ® tocopherol acetate | (1) | TOCOPHERYL ACETATE | 0.20 |

Preparation:

Weigh out all constituents of the phase together and grind homogeneously in a mixer. Subsequently add phase C and continue mixing, then add phase A and grind briefly until the pigments are uniformly distributed.

Sources of Supply:

(1) Merck KGaA/Rona®

(2) Cognis GmbH

(3) Sasol Germany GmbH

1. Cosmetic formulations, characterised in that they comprise, as light-protection filters in the wavelength region >700 nm, at least one substantially colour-neutral, transparent multilayered pigment based on flake-form substrates.

2. Cosmetic formulations according to claim 1, characterised in that the multilayered pigment has
(A) a colourless coating having a refractive index of n>1.8 and a layer thickness of 50-350 nm,
(B) a colourless coating having a refractive index of n≦1.8 and a layer thickness of 50-500 nm,
(C) a colourless coating having a refractive index of n>1.8 and a layer thickness of 20-350 nm.

3. Cosmetic formulations according to claim 1, characterised in that the substrate comprises synthetic or natural mica flakes, SiO₂ flakes, glass flakes, Al₂O₃ flakes, polymer flakes or mixtures thereof.

4. Cosmetic formulations according to claim 1, characterised in that layer (A) consists of TiO₂, ZrO₂, SnO₂, Fe₂O₃, Cr₂O₃, ZnO, BiOCl or mixtures or combinations thereof.

5. Cosmetic formulations according to claim 1, characterised in that layer (B) consists of SiO₂, MgF₂, B₂O₃, AlO(OH), MgSiO₃ or Al₂O₃ or mixtures or combinations thereof.

6. Cosmetic formulations according to claim 1, characterised in that layer (A) and layer (C) have the same composition.

7. Cosmetic formulations according to claim 1, characterised in that layer (A) and layer (C) consist of TiO₂.

8. Cosmetic formulations according to claim 1, characterised in that the two TiO₂ layers have identical or different layer thicknesses.

9. Cosmetic formulations according to claim 1, characterised in that the multilayered pigment additionally contains a ZnO layer between layers (A) and (B) and/or between layers (B) and (C) and/or on layer (C) and/or on the substrate.

10. Cosmetic formulations according to claim 1, characterised in that layers (A), (B) and/or (C) may additionally comprise finely divided particles.

11. Cosmetic formulations according to claim 1, characterised in that the finely divided particles are TiO₂, ZnO, carbon or carbon black.

12. Cosmetic formulations according to claim 1, characterised in that the multilayered pigment has a C value of <30 (measured using an ETA Plus calorimeter).

13. Cosmetic formulations according to claim 1, characterised in that the multilayered pigment is present in amounts of 0.5-100% by weight, based on the formulation.

14. Cosmetic formulations according to claim 1, characterised in that they additionally comprise fillers and colorants.

15. Cosmetic formulations according to claim 1, characterised in that the colorant is flake-form, spherical or needle-shaped.

16. Cosmetic formulations according to claim 1, characterised in that the colorant is an organic or inorganic coloured pigment or an organic dye.

17. Cosmetic formulations according to claim 1, characterised in that, besides the multilayered pigment, the formulation comprises at least one constituent selected from the group absorbents, astringents, antimicrobial substances, antioxidants, antiperspirants, antifoaming agents, antidandruff active compounds, antistatics, binders, biological additives, bleaches, chelating agents, deodorants, emollients, emulsifiers, emulsion stabilisers, colorants, dyes, humectants, film formers, fillers, fragrances, flavours, insect repellents, preservatives, corrosion-protection agents, cosmetic oils, solvents, oxidants, vegetable constituents, buffer substances, reducing agents, surfactants, propellant gases, opacifiers, UV filters, UV absorbers, denaturing agents, viscosity regulators, perfume and vitamins.

18. Cosmetic formulations according to claim 1, characterised in that they additionally comprise a UV filter.

19. Cosmetic formulations according to claim 1, characterised in that they are lipophilic, hydrophilic or hydrophobic.

20. Use of the cosmetic formulations according to claim 1 in decorative and care cosmetics.

21. A method of protecting skin from light comprising administering a formulation of claim 1 as a decorative or care cosmetic.
